(19) United States
(12) Patent Application Publication
(10) Pub. No.: US 2012/0073572 A1
(43) Pub. Date: Mar. 29, 2012

(54) Intubation Stylet & Endotracheal Tube

(76) Inventor: Michael Y. Li, Syosset, NY (US)

(21) Appl. No.: 12/890,213

(22) Filed: Sep. 24, 2010

(51) Int. Cl. A61M 16/00

(52) U.S. Cl. 128/200.26

(57) ABSTRACT

An intubation stylet apparatus for use in medical intubation of a patient where the stylet includes an elongated tube having a distal end section, a proximal end section and an intermediate section located between the distal end section and the proximal end section. A grip which is attached to the proximal end section is coupled to a control lever which is attached to the ends of at least two control wires and the other ends of the control wires are attached to the distal end of the elongated tube. In operation, the distal end of the elongated tube is urged to bend to a modified “S” shaped curve which resembles the body, neck and head of a rattle sneak when in its raised position when the control lever is pulled to apply a tension force to the wires, and to return to a straight tube when the tension force on the wires is released.

### BACKGROUND OF THE INVENTION

1. Field of the Invention

The present invention relates to an intubation tube and more particularly, to an intubation apparatus having a stylet that includes a distal end that has a flexible tip which is connected through a intermediate member to a syringe like grip for use in placing an endotracheal tube into the trachea of a patient. The flexible tip of the stylet is connected with two fine wires to a spring loaded control member which is slidably coupled to the syringe like grip. Applying tension to the wires by sliding the spring loaded control member along the syringe like grip urges the tip of the stylet to bend at a desired amount and in a desired direction.

2. Description of Related Art

Stylets of Intubation devices, such as oral-endotracheal tubes which are used in a wide variety of medical situations to provide an unobstructed conduit to a patient's trachea in which oxygen, medications and therapies can be passed are known in the prior art. More specifically, by way of example, U.S. PreGrant Publication No. 2010/0108060 to Pecherer discloses an articulated stylet member with a link at its distal end, a L-shaped lever with a downward depending handle for tautening a flexible wire for flexing the link into a flexed positioned to form a hook-like configuration in the stylet member's side view, and a stopper mechanism mounted on the stylet member for stopping insertion of the stylet member into an endotracheal tube at a suitable position depending on the endotracheal tube's length.

U.S. PreGrant Publication No. 2006/0095018 to Pursley discloses a catheter having a curved distal end in which a resilient fiber embedded in a polymer material of the sidewall imparts a bend in the catheter. The resilient fiber has a helical coil shape with a series of helical coils disposed about a center line.

U.S. Pat. No. 6,705,989 to Cuschieri discloses a retractor for use in endoscopic surgery, having an adjustable distal end which consists of several link elements that can be moved into a ring-shaped structure and the ring-shaped structure can be bent in relation to the shaft in a continuous manner at an angle of up to 90 degrees.

U.S. Pat. No. 5,882,346 to Pomeranz discloses a core wire which includes a pre-shaped region bent into a predetermined shape. A catheter is provided which includes a lumen proportioned to slidably receive the core wire and includes a rigid proximal section and a flexible distal section.

U.S. Pat. No. 5,749,357 to Linder discloses a catheter having a malleable tube with a smooth, inflatable introducer tip for guiding the catheter. The malleable tube is made of a ductile metal such as aluminum or copper.

U.S. Pat. No. 5,362,734 to Galel discloses a catheter having a tubular member which contains a series of relatively rigid segments alternating with flexible, collapsible segments which permits the rigid segments to effectively stack against one another when the tubular member is compressed axially.

U.S. Pat. No. 5,176,126 to Chikama discloses a bending device for use in an endoscope having a frame of a generally cylindrical shape, a limitation member and an operating wire. The frame has annular portions disposed respectively in planes substantially perpendicular to an axis of the frame, and the annular portions are juxtaposed in an axial direction of the frame. The annular portions of the frame are engaged in the engaging recesses, so that any adjacent ones of the annular portions are spaced a predetermined distance from each other.

U.S. Pat. No. 3,996,939 to Sheridan discloses a stylet having a bendable metal rod hermetically sealed in a tubular plastic sheath where the rod is closely encircled by the sheath where the two can flex longitudinally independently of each other.

### SUMMARY OF THE INVENTION

In an exemplary embodiment of the present invention, there is disclosed an intubation stylet apparatus for use in medical intubation of a patient where the stylet includes an elongated tube having a distal end section, a proximal end section and an intermediate section located between the distal end section and the proximal end section. A grip which is attached to the proximal end section is coupled to a control lever which is attached to the ends of at least two control wires and the other ends of the control wires are attached to the distal end of the elongated tube. In operation, the distal end of the elongated tube is urged to bend to a modified “S” shaped curve which resembles the body, neck and head of a rattle sneak when in its raised position when the control lever is pulled to apply a tension force to the wires, and to return to a straight tube when the tension force on the wires is released.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a side view showing a stylet that includes a distal end that has a flexible tip which is connected through an intermediate member of semi flexible tubing to a syringe like grip for use in placing an endotracheal tube into the trachea of a patient. The flexible tip of the stylet is connected with two fine wires to a spring loaded control member which is slidably coupled to the syringe like grip. Increasing the tension on the wires by sliding the spring loaded control member along the syringe like grip urges the flexible tip of the stylet to bend a desired amount either forward or backward to orient the flexible tip of the stylet in a desired direction.

FIG. 2 is a side view of an elongated endotracheal tube having a proximate open end, a distal open end and a intermediate tube located between the distal and proximate ends;

FIG. 3 is a side view of the endotracheal tube of FIG. 2 located around the stylet of FIG. 1;

FIG. 4 is a side sectional view of the flexible tip where the distal and proximate ends of each segment of the flexible tip are flat and converge toward each other to form a segment having a trapezoid shape and the top, bottom and sides of each segment can be curved;

FIG. 5 is an end view of the flat end of a segment showing the openings for receiving control wires;

FIG. 6 is a view of the motion obtained with a prior art stylet; and

FIG. 7 is a view of the motion obtained with the stylet here disclosed.

### DESCRIPTION OF THE PREFERRED EMBODIMENT

Intubation is the placement of a tube into the trachea of a person who is unconscious or unable to breathe on his/her own to maintain an open airway through which oxygen and/or anesthetics can be supplied.

The most common path for the tube is through the oropharynx, glottis, and larynx and into the trachea. After the endotracheal tube is positioned, a low-pressure cuff is inflated near the distal tip of the tube to help secure it in place and protect the airway from blood and gastric fluids. The tube can then be secured to the face or neck of the patient, and the proximal end of the tube may be connected to a breathing apparatus or the like.

The basic technique of inserting a tube into a person usually involves opening the patient's mouth by separating his/her jaws to allow a stylet to be inserted into the mouth. The stylet is inserted into the patient's mouth with the tip of the stylet being moved toward the right tonsil. Upon reaching the right tonsil, the stylet is swung to the center of the mouth making sure that the patients tongue is on the left to allow the epiglottis to be seen. The tip of the stylet is then moved forward until it reaches the junction between the base of the tongue and the epiglottis. At this time the stylet is lifted up towards the chest to expose the vocal cords and the cuff of the endotracheal tube, which is located below the vocal cords, is inflated.

Thus, inserting a tube into a person is an invasive procedure that requires the distal end of the stylet to be moved in various directions by moving the distal end of the stylet from a location which is many inches back from the distal end of the tube. The procedure of pushing and pulling the distal end of the stylet forward and backward from a location which is inches back from the distal end, if not done properly may result in serious harm to the patient.

The present invention relates to a new improved apparatus and method of performing endotracheal intubation. As used in the description, the term “distal” is intended to refer to the direction towards the patient receiving the intubation device, and the term “proximal” is intended to refer to the direction away from the patient receiving the intubation device.

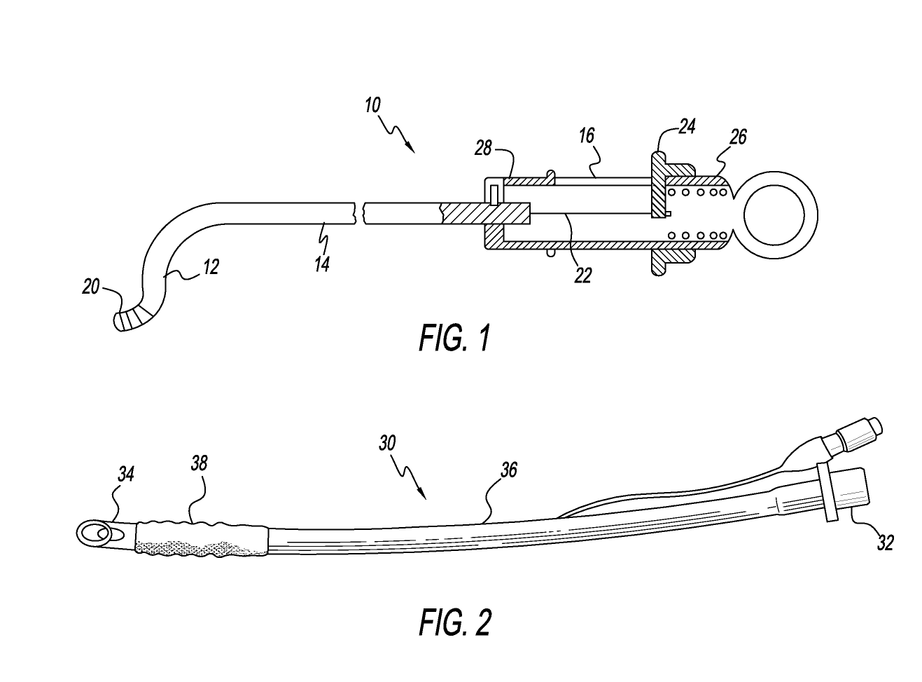

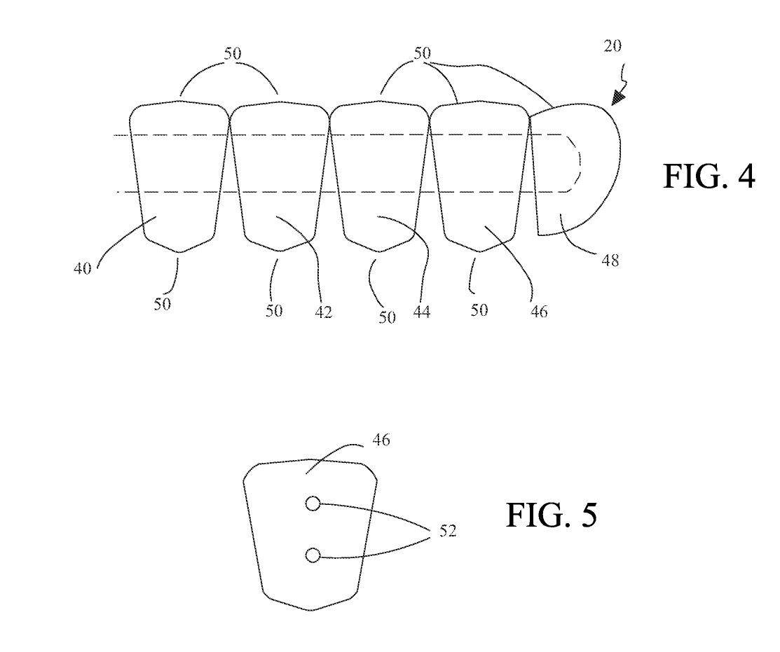

Referring to FIG. 1, there is shown an elongated stylet 10 which has a tip 12 which is formed from plurality of segments such as between three and seven where between three or four segments is preferable. The distal and proximate ends of each segment of the flexible tip are flat and converge toward each other to form segments having a trapezoid shape (see FIG. 4) and where the top, bottom and sides of each segment can be curved. Each segment is composed of a flexible material that can be bent relatively easily into a desirable contour. The tip 12 is attached to an intermediate tube 14 of semi-flexible material which is attached to a grip such as a syringe like cylindrical member 16 which has a spring loaded pull control lever 24 slidably coupled to the outside surface of the syringe like grip. The flexible tip 12 can be composed of polyethylene, polypropylene or a plastic where the composition of the material is controlled to insure that the tip 12 is flexible, and the semi-flexible intermediate tube 14 can be made of a semi-flexible plastic or of stainless steel. If desired the proximal end of the intermediate tube can have a larger cross section. The most distal end 20 of the tip 12 is preferably rounded or tapered to minimize injury to the tissue of the patient during intubation.

Referring to FIG. 4, there is shown a side sectional view of the flexible tip 20 where the distal and proximate ends of each segment of the flexible tip are flat and converge toward each other to form a segment having a trapezoid shape and the top, bottom and sides of each segment can be curved. The top, bottom, and sides of each segment 40, 42, 44, 46, 48 of the flexible tip can have a cylindrical, curved or convex shape 50. The convex shape at the end of the most digital segment 48 has a circular shape with a generous radius.

Referring to FIG. 5, there is shown an end view of the flat proximate end of a segment, for example segment 46. Located along a center line of the segment are two openings 52 for receiving control wires.

The flexible end 20 of the distal tip 12 is attached to two small diameter control wires 22 which can be of stainless steel or the like with a diameter of about fifteen thousands of an inch more or less. The wires extend from the distal end 20, through the openings 52 in the segments of the flexible end 20 of the flexible tip 12, through the intermediate tube 14 to the spring loaded pull control lever 24. The spring 26 which is in compression urges the control lever to move toward the left. The flexible end 20 springs back to a straight tube when the control wires are not in tension but bend as tension is applied to the control wires by pulling on the control lever. Thus, when the spring 26 is not compressed and the control lever is located at the left end 28 of the grip 16, the control wires will be in their relaxed condition and the flexible end 20 will be in its unstressed straight position and will be aligned with the straight intermediate tube. But, as the control lever is slowly moved toward the right and compresses the spring 26, the control wires will urge the segments of the flexible end 20 to bend. As the control lever is moved further along the grip to further compress the spring, the bending of the flexible end will increase. The direction in which the flexible end bends can be controlled by moving the control lever along the grip.

Referring to FIG. 2, there is shown an endotracheal tube 30 having an opening at its proximal end 32 and an opening at its distal end 34. An intermediate tube 36 is located between the proximal and distal ends. Tube 36 can be formed of a semi-rigid material such as polyvinyl chloride, silicone rubber or stainless steel and has an inflatable cuff 38 located slightly back from the distal end 34. The inflatable cuff is attached to a small plastic tube which, when connected to a source of air pressure such as a syringe, will inflate the cuff to seal the trachea and bronchial tree against leakage of respiratory gases and fluids.

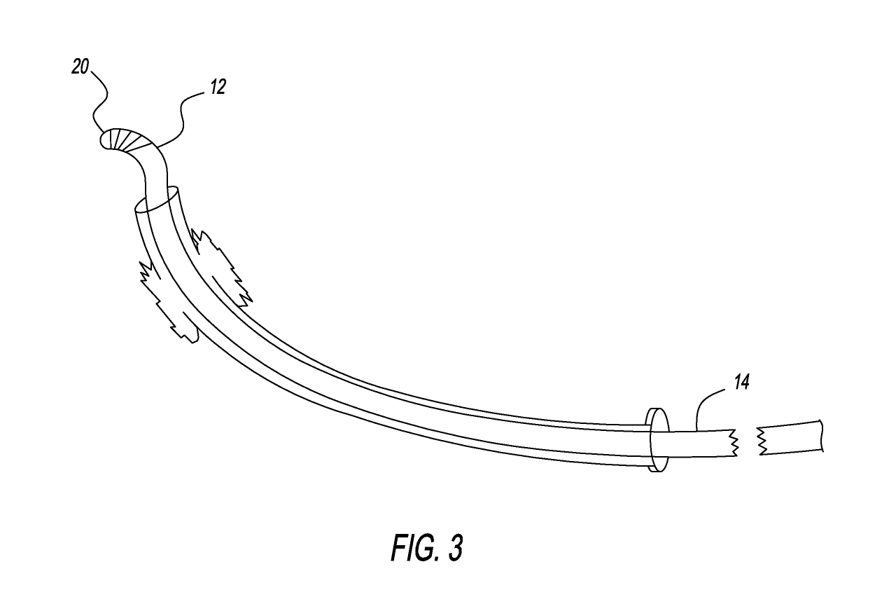

Referring to FIG. 3 there is shown a side view of the endotracheal tube of FIG. 2 located around the stylet of FIG. 1. Most endotracheal tubes are constructed of polyvinyl chloride. However tubes of wire-reinforced plastic or stainless steel are available for special applications. The inside diameter of the tubes can be between 2 mm and 10.5 mm where the size that is used depends on the patient's body size. Endotracheal tubes have an inflatable cuff to seal the trachea and bronchial tree against leakage of respiratory gases and aspiration of gastric contents, blood, secretions, and other fluids.

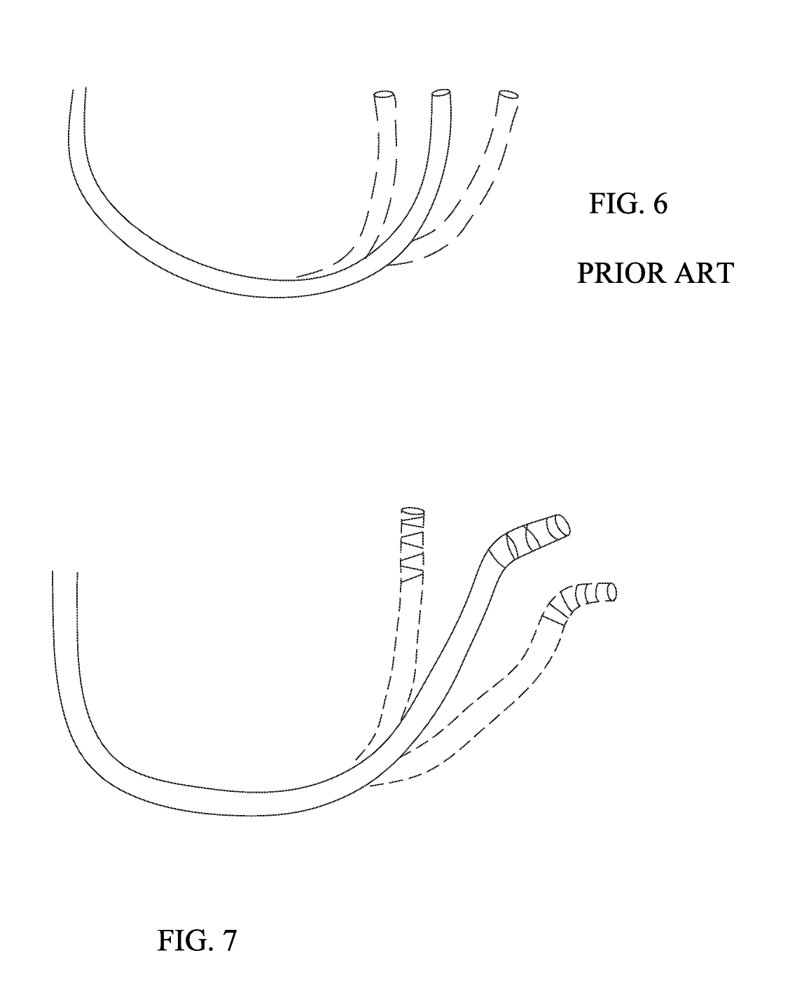

Referring to FIG. 6, Fig. there is shown a view of the motion obtained with a prior art stylet; and looking at FIG. 7, there is shown a view of the motion obtained with the stylet here disclosed.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. An intubation stylet apparatus for use in medical intubation of a patient, the stylet comprising:
an elongated tube having a distal end section, a proximal end section and an intermediate section located between the distal end section and the proximal end section;
a grip attached to the proximal end section;
a control lever slidably coupled to the grip; and
wires having one end attached to the control lever and the other end attached to the distal end of the elongated tube;
wherein the distal end of the elongated tube has at least three trapezoid shaped cylindrical segments, each segment, other than the most distal segment, having distal and proximate ends that are flat and the distal end of the most distal segment is a semi circle, the distal end of the elongated tube being urged to bend to a modified “s” shaped curve when the control lever is pulled to apply a tension force on the wires and to return to a straight tube when the tension force on the wires is released; and
wherein the distal end segment and the intermediate segments are composed of polyethylene, polypropylene or a plastic composition where the composition of the material is controlled to insure that the distal end of the elongated tube is more flexible than the intermediate section.

2. The intubation stylet apparatus of claim 1 wherein the control lever is coupled to a spring which urges the control lever to move in a direction which reduces the tension in the control wires.

3. The intubation stylet apparatus of claim 2 wherein the distal end section is made of a flexible plastic and the intermediate section is made of a semi-flexible plastic or of stainless steel.

4. The intubation stylet apparatus of claim 3 wherein the control wires are located within the proximal end section, the intermediate section and the distal end section.

5. The intubation stylet apparatus of claim 4 wherein the control wires includes two spaced apart wires.

6. The intubation stylet apparatus of claim 5 wherein the bend of the distal end of the elongated tube increases as the pull on the control lever is increased to apply an increased tension force on the wires.

7. The intubation stylet apparatus of claim 6 wherein the end of the distal end of the elongated tube is rounded or tapered to minimize injury to the tissue of the patient during insertion.

8. The intubation stylet apparatus of claim 7 wherein the wires are made of stainless steel.

9. The intubation stylet apparatus of claim 8 wherein the grip is a cylindrical member and the spring is located within the cylindrical member.

10. A method of medically intubating a patient comprises: providing a stylet having a distal end section, a proximal end section and an intermediate section located between the distal end section and the proximal end section;
attaching a grip to the proximal end section of the stylet;
slidably coupling a control lever to the grip;
providing wires having one end attached to the control lever and the other end attached to the distal end of the elongated tube;
inserting the distal end section, proximal end section and intermediate section of the stylet into an endotracheal tube; and
inserting the stylet and endotracheal tube into the trachea of the patient;
wherein as the stylet and endotracheal tube is being inserted into the trachea the distal end of the elongated tube is urged to bend when the control lever is pulled to apply a tension force on the wires and to return to a straight tube when the tension force on the wires is released; and
wherein the distal end section and the intermediate section are composed of polyethylene, polypropylene or a plastic composition where the composition of the material is controlled to insure that the distal end section is more flexible than the intermediate section.

11. The method of claim 10 wherein the control lever is coupled to a spring which urges the control lever to move in a direction which reduces the tension in the control wire.

12. The method of claim 11 wherein the distal end section is made of a flexible plastic and the intermediate section is made of a semi-flexible plastic.

13. The method of claim 12 wherein the wires are located within the proximal end section, the intermediate section and the distal end section.

14. The method of claim 13 wherein the wires includes two spaced apart wires.

15. The method of claim 14 wherein the bend of the distal end of the elongated tube increases as the pull on the control lever is increased to apply an increased tension force on the wires.

16. The method of claim 15 wherein the end of the distal end of the elongated tube is rounded or tapered to minimize injury to the tissue of the patient during insertion.

17. The method of claim 16 wherein the wires are made of stainless steel.

18. The method of claim 17 wherein the grip is a cylindrical member and the spring is located within the cylindrical member.
